(19) United States
(12) Patent Application Publication
(10) Pub. No.: US 2011/0068191 A1
(43) Pub. Date: Mar. 24, 2011

(54) DECORATIVE AROMA DIFFUSER

(76) Inventor: CARSTEN JÖRGENSEN, KASTANIENBAUM (CH)

(21) Appl. No.: 12/566,302

(22) Filed: Sep. 24, 2009

(51) Int. Cl. A61L 9/00; B05B 1/24

(52) U.S. Cl. 239/34; 239/135

(57) ABSTRACT

A decorative aroma diffuser includes a ceramic bowl-like tray for holding an aromatic fluid and flower petals/herb leaves, the tray having endless ribs concentrically raised from the inside wall and the outside wall to reinforce the structural strength, a base fastened to the bottom side of the tray and defining an accommodation chamber, a heater mounted in the accommodation chamber for heating the aromatic fluid into vapor, and a bottom plate fastened to the bottom side of the base and carrying a power source that provides electricity to the heater.

### BACKGROUND OF THE INVENTION

1. Field of the Invention

The present invention relates to aroma diffusers and more particularly, to a decorative aroma diffuser that provides a decorative function.

2. Description of the Related Art

People who are sensitive to beauty do care about life style and personal taste. Using an aroma diffuser in the house can create a unique and peaceful atmosphere.

Aroma diffusing has been used in many chain hotels as a unique service to attract clients. By means of the central air conditioning system, aroma is diffused into the air being delivered to every room of the building. Different aromatic smells may be released in different seasons to create different atmosphere. For example, teat tree aroma with peppermint aroma may be applied in summer; orange essential oil may be used in winter. However, a big-scale aroma diffusing system for use in hotels is not practical for home application.

There are small aroma diffusers practical for home use. These aroma diffusers commonly have a housing made from ceramics. However, if the ceramic housing has a thin wall thickness, it may break easily. If the ceramic housing has a thick wall, it does not facilitate transfer of heat energy from the heat source mounted therein to the aromatic fluid held on its top side. Further, if the diffusing mouth of the fluid chamber at the top side of the ceramic housing is narrow, it will take a long time to start diffusing of the aromatic smell after heating of the aromatic fluid by the heater. Further, conventional aroma diffusers do not facilitate cleaning. Further, regular aroma diffusers are not attractive when used as a decorative item.

### SUMMARY OF THE INVENTION

The present invention has been accomplished under the circumstances in view. It is main object of the present invention to provide a decorative aroma diffuser, which provides a decorative effect and facilitates cleaning. It is another object of the present invention to provide a decorative aroma diffuser, which is safe in use. It is still another object of the present invention to provide a decorative aroma diffuser, which saves power consumption.

To achieve these and other objects of the present invention, a decorative aroma diffuser comprises a tray, a base, a heater and a bottom plate. The tray is made from ceramics in the shape of a bowl for holding an aromatic fluid, comprising a top side curved inwards and defining a holding space for holding an aromatic fluid, a bottom side, and a plurality of endless ribs respectively raised from the top side and the bottom side to reinforce the structural strength. The base is fixedly connected to the bottom side of the tray, thereby defining an accommodation chamber. Further, the base comprises two inside flanges bilaterally suspending in the accommodation chamber and facing each other. The heater is mounted in the accommodation chamber and kept in contact with the bottom side of the tray and adapted to heat the tray. The bottom plate is covered on the base to close the accommodation chamber. Further, the bottom plate carries a power source for providing electricity to the heater to heat the tray.

The decorative aroma diffuser further comprises an aromatic fluid carried in the holding space in the tray, and at least one flower petal or one herb leaf carried on the aromatic fluid. Further, the heater is a cement resistor for heating the aromatic fluid in the tray at a temperature within the range about 15° C.˜60° C. This low heating temperature does not cause the herb leaves or flower petals to be dissolved out quickly.

The decorative aroma diffuser further comprises a locating member supported between the two inside flanges inside the base, and a fastening device fastened to the locating member to lock the heater to the bottom side of the tray. Further, the locating member comprises a mounting through hole. Further, the fastening device comprises a tightening-up screw threaded into the mounting through hole of the locating member and stopped at one side of the heater against the bottom side of the tray, and a nut threaded onto the tightening-up screw and stopped at the bottom side of the bottom plate. Further, each inside flange of the base comprises a locating groove. Further, the locating member has two opposite ends respectively fastened to the locating grooves of the two inside flanges of the base. Further, the two inside flanges and the base are formed integral with the tray. Further, the bottom plate comprises a plurality of foot members equiangularly disposed at a bottom side thereof.

### BRIEF DESCRIPTION OF THE DRAWINGS

FIG. 1 is an exploded, oblique bottom view of a decorative aroma diffuser in accordance with the present invention.

FIG. 2 is an exploded, oblique top view of the decorative aroma diffuser in accordance with the present invention.

FIG. 3 is a sectional assembly view of the decorative aroma diffuser in accordance with the present invention.

FIG. 4 is a schematic applied view of the present invention, showing an aromatic fluid and flower petals carried in the tray.

FIG. 5 is another schematic applied view of the present invention, showing an aromatic fluid and herb leaves carried in the tray.

### DETAILED DESCRIPTION OF THE PREFERRED EMBODIMENT

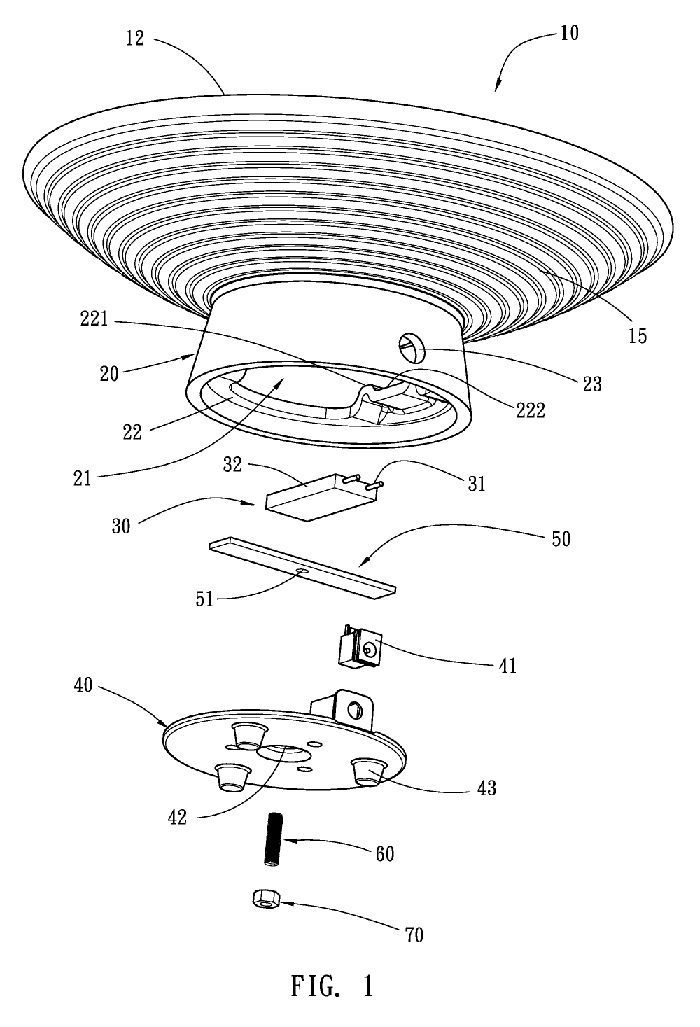

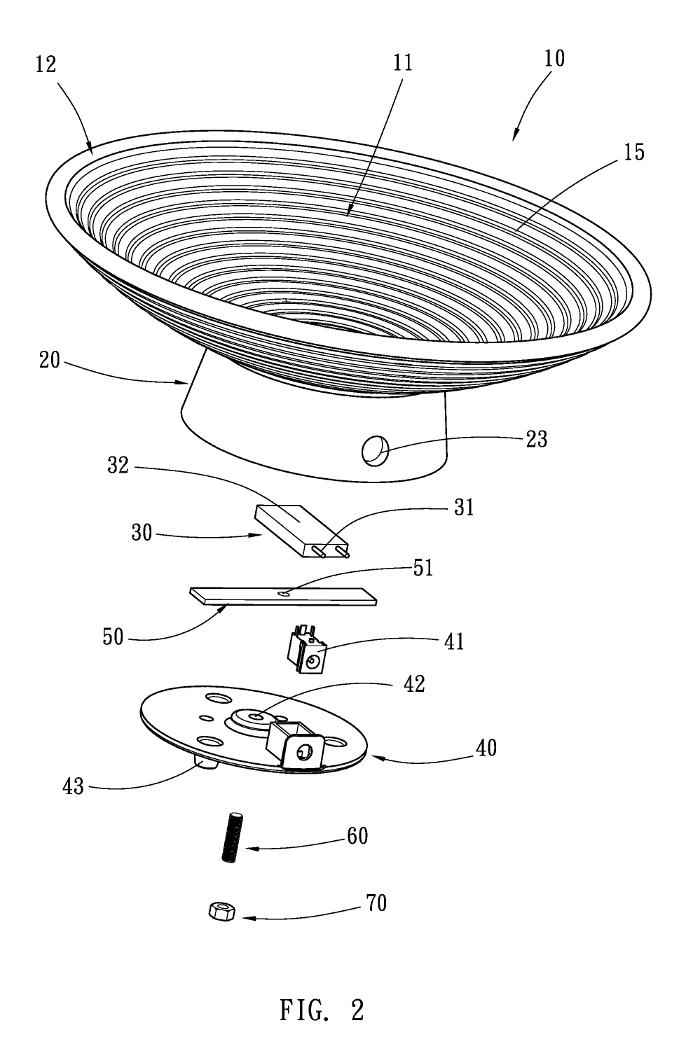

Referring to FIGS. 1 and 2, a decorative aroma diffuser in accordance with the present invention is shown comprising a tray 10, a base 20, a heater 30 and a bottom plate 40.

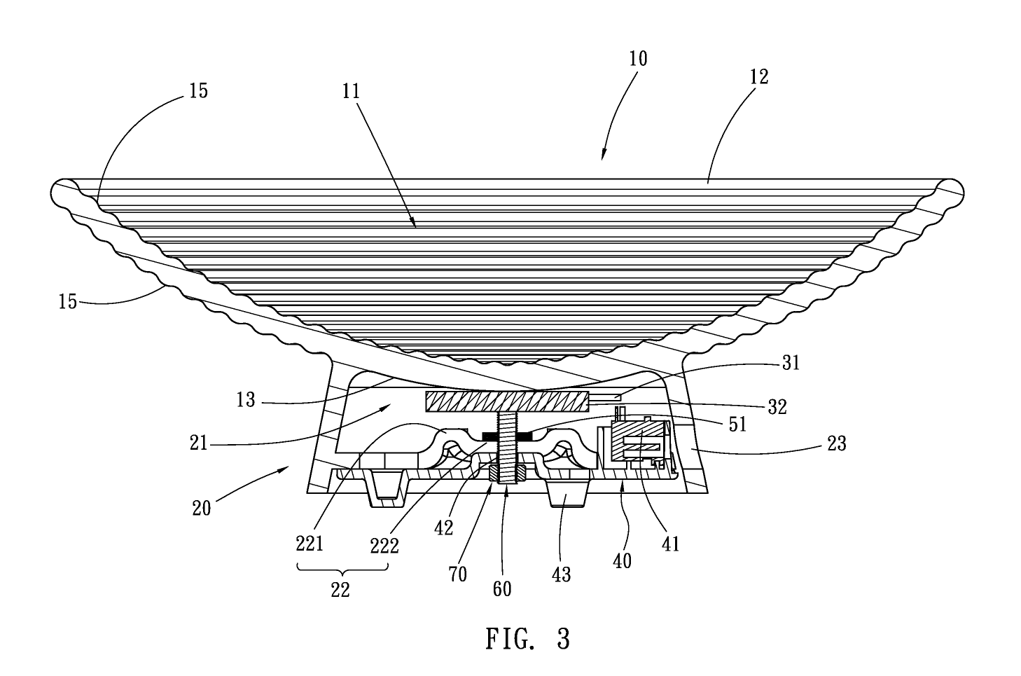

Referring to FIG. 3, the tray 10 is prepared from ceramics and shaped like a bowl, having a relatively wider top side 12 and a relatively narrower bottom side 13 and defining a holding space 11 for holding a fluid. The tray 10 further comprises a plurality of endless ribs 15 concentrically arranged on the inside and outside walls thereof to reinforce the structural strength and to increase the fluid contact surface area in the holding space 11, thereby enhancing thermal transfer and warm keeping effects. Further, the endless ribs 15 generate an image that simulates waves and ripples of water to attract people's eyes.

The base 20 is a hollow member prepared from the same material used for the tray 10, and formed integral with the bottom side 13 of the tray 10. The base 20 defines with the bottom side 13 of the tray 10 an accommodation chamber 21. Further, the base 20 comprises two inside flanges 22 suspending in the bottom open side of the accommodation chamber 21 and opposing each other. Each inside flange 22 has two protruding portions 221 and a locating groove 222 defined between the two protruding portions 221.

The heater 30 is a cement resistor. Further, the heater 30 is mounted in the accommodation chamber 21 and kept in contact with the bottom side 13 of the tray 10 for generating heat to heat the tray 10.

The bottom plate 40 is mounted on the bottom side of the base 20, carrying a power source 41 and having a mounting through hole 42. The power source 41 provides electricity to the heater 30, causing the heat source 30 to generate heat in heating the tray 10. According to the present preferred embodiment, the heat source 41 is a power jack.

Further, the heater 30 is a flat member having two power contacts 31 electrically connected to the power source 41 and one broad side 32 kept in contact with the bottom side 13 of the tray 10.

The decorative aroma diffuser further comprises a locating member 50 and a fastening member 60. The locating member 50 is a metal bar, preferably iron bar. The fastening member 60 is a metal tightening-up screw. The locating member 50 is bridging the gap between the two inside flanges 22 of the base 20, having two opposite ends respectively engaged into the locating groove 222 on each of the two inside flanges 22. The fastening member 60 is threaded into the mounting through hole 42 of the bottom plate 50 and stopped at the bottom side of the heater 30 against the tray 10.

After installation of the fastening member 60 in the mounting through hole 42 of the bottom plate 50 to lock the heater 30 to the tray 10, a nut 70 is threaded onto the end of the fastening member 60 and stopped at the bottom side of the bottom plate 40.

The bottom plate 40 has foot members 43 equiangularly disposed at the bottom side for supporting the whole assembly of the decorative aroma diffuser on a flat surface stably.

When comparing to the conventional designs that have drawbacks, such as cleaning inconveniency, thick wall thickness of ceramic housing to lower thermal transfer efficiency from the heater to the applied aromatic fluid, fragile characteristic of thin wall thickness of ceramic housing or low diffusing speed at the initial stage due to narrow diffusing mouth of the ceramic housing and the use of a low temperature heater, the decorative aroma diffuser has the characteristics of high strength, excellent heat transfer and warm keeping effects and power saving. Subject to the shape design of the open type tray 10 and the arrangement of the endless ribs 15, the aroma diffuser provides a decorative effect and facilitates cleaning. The open type holding space 11 of the tray 10 facilitates filling and diffusing of aroma fluid. The endless ribs 15 reinforce the structural strength of the tray 10 so that the tray 10 does not break easily and facilitates transfer of heat energy from the heater 30 to the aroma fluid held in the holding space 11. Because of high thermal transfer efficiency, the invention saves consumption of power supply. As the endless ribs 15 are raised from the inside and outside walls of the tray 10 and arranged in a concentric manner, they impart an image that simulates waves and ripples of water to attract people's eyes. As the cement resistor type heater 30 is kept in contact with the bottom side of the tray 10, heat energy generated by the heater 30 is directly transferred to the tray 10, assuring a high level of safety. The bowl-shaped tray 10 facilitates application of an aromatic fluid. Further, herb leaves or flower petals can be placed on the aromatic fluid in the tray 10 for decoration, creating an atmosphere of nice feeling. Further, using the open type tray 10 to carry an aromatic fluid facilitates diffusing of the aromatic fluid and cleaning of the diffuser.

Further, because the invention utilizes a cement resistor to generate heat for heating the applied aromatic fluid at a low temperature about within the range about 15° C.˜60° C., slow evaporation of the aromatic fluid does not causes the herb leaves or flower petals will not be dissolved out quickly.

The inside flanges 22, the base 20 and the tray 10 are made from ceramics in integrity, having high strength and electric insulation characteristics. Therefore, the decorative aroma diffuser does not break easily.

Because the invention utilizes a cement resistor to heat the aromatic fluid at a low or medium temperature, touching the tray 10 with the hands does not cause the hands to be scalded, assuring safety. When heating the aromatic fluid into vapor, a pleasant smell is diffused into the air without causing the herb leaves or flower petals to be dissolved out quickly.

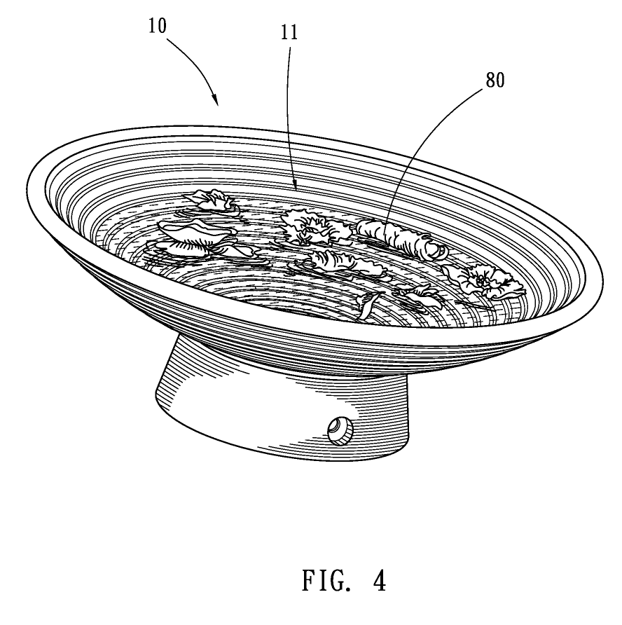

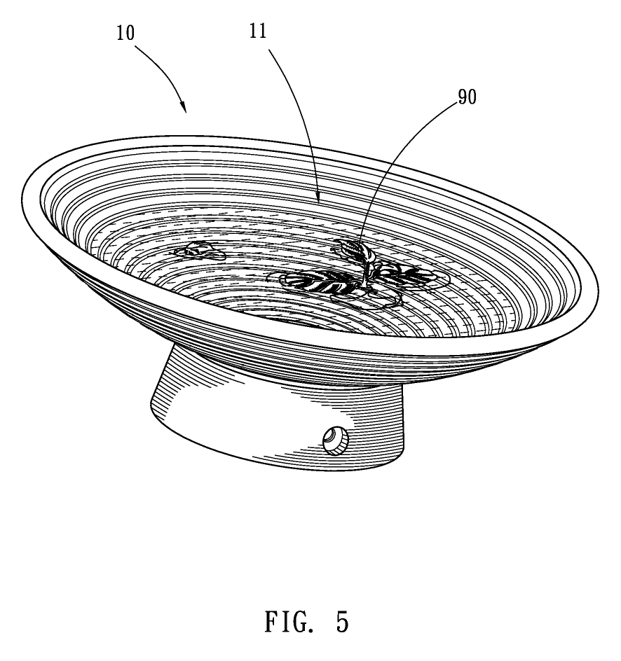

When using the decorative aroma diffuser, an aromatic fluid is filled in the holding space 11 of the tray 10, and then flower petals 80 (see FIG. 4) or herb leaves, for example, peppermint leaves 90 (see FIG. 5) are add to the aromatic fluid. The aromatic fluid is heated into vapor slowly. Different flower petals or different herb leaves may be selectively added to the aromatic fluid subject to one's preference, providing a decorative effect and giving a certain pleasant smell.

Further, adding different herb leaves to essential oil creates different smells. For example, rose petals can be added to lavender essential oil for diffusing a special pleasant smell that is good for the respiratory system and the integument system.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A decorative aroma diffuser, comprising:
a tray made from ceramics in the shape of a bowl for holding an aromatic fluid, said tray comprising a top side curved inwards and defining a holding space for holding an aromatic fluid, a bottom side, and a plurality of endless ribs respectively raised from said top side and said bottom side;
a base fixedly connected to the bottom side of said tray, said base defining with the bottom side of said tray an accommodation chamber, said base comprising two inside flanges bilaterally suspending in said accommodation chamber and facing each other;
a heater mounted in said accommodation chamber and kept in contact with the bottom side of said tray and adapted to heat said tray; and
a bottom plate covered on said base to close said accommodation chamber, said bottom plate carrying a power source for providing electricity to said heater to heat said tray.

2. The decorative aroma diffuser as claimed in claim 1, further comprising an aromatic fluid carried in said holding space in said tray, and at least one of flower petals and or leaves carried on said aromatic fluid.

3. The decorative aroma diffuser as claimed in claim 1, further comprising a locating member supported between said two inside flanges inside said base, and a fastening device fastened to said locating member to lock said heater to the bottom side of said tray.

4. The decorative aroma diffuser as claimed in claim 1, wherein said heater is a cement resistor.

5. The decorative aroma diffuser as claimed in claim 3, wherein each said inside flange of said base comprises a locating groove; said locating member has two opposite ends respectively fastened to the locating grooves of said two inside flanges of said base.

6. The decorative aroma diffuser as claimed in claim 1, wherein said two inside flanges and said base are formed integral with said tray.

7. The decorative aroma diffuser as claimed in claim 1, wherein said bottom plate comprises a plurality of foot members equiangularly disposed at a bottom side thereof.

8. The decorative aroma diffuser as claimed in claim 3, wherein said locating member comprises a mounting through hole; said fastening device comprises a tightening-up screw threaded into said mounting through hole of said locating member and stopped at one side of said heater against the bottom side of said tray, and a nut threaded onto said tightening-up screw and stopped at the bottom side of said bottom plate.
